(19) United States
(12) Patent Application Publication
(10) Pub. No.: US 2025/0088071 A1
(43) Pub. Date: Mar. 13, 2025

(54) GYROSCOPIC MECHANICAL AND ELECTRICAL BATTERY FOR STABILIZING A SURGICAL ROBOTIC MOBILE CART

(71) Applicant: Covidien LP, Mansfield, MA (US)

(72) Inventors: Ulrich Hagn, Muenchen (DE); Jaimeen V. Kapadia, Cambridge, MA (US)

(21) Appl. No.: 18/705,637

(22) PCT Filed: Nov. 2, 2022
(86) PCT No.: PCT/IB2022/060559

§ 371 Date: Apr. 29, 2024

Related U.S. Application Data
(60) Provisional application No. 63/274,970, filed on Nov. 3, 2021.

(51) Int. Cl. H02K 7/02; A61B 34/30; A61B 50/13; B25J 5/00; B62D 37/06; F03G 3/08

(52) U.S. Cl. CPC H02K 7/025; A61B 34/30; A61B 50/13; B25J 5/007; B62D 37/06; F03G 3/083

(57) ABSTRACT

A flywheel energy storage device is included on a mobile robotic cart or a robotic arm to stabilize the cart and/or arm as well as provide electric energy allowing for uninterrupted power supply thereto.

### BACKGROUND

Surgical robotic systems may include a surgical console controlling one or more surgical robotic arms, each having a surgical instrument having an end effector (e.g., forceps or grasping instrument). In operation, one or more robotic arms are moved to a position over a patient and the surgical instrument is guided into a small incision via a surgical access port or a natural orifice of a patient to position the end effector at a work site within the patient's body. The robotic arm may be mounted with its base on a mobile cart, a surgical table, a ceiling support system, etc. Typically, such systems are supplied with electric energy for operation and maintaining position stability ensures the precision of the system.

The robotic arms may be supplied with electric energy using cables interconnecting the robotic arms to electric mains. Depending on the mount type (e.g., a mobile cart, a surgical table, a ceiling support system, etc.) of the robotic arms, the cables may need to be integrated into and around the mount locations. The electrical cables may cause tripping hazards, clutter the space around the surgical table, and block motion of other devices.

### SUMMARY

This disclosure provides a combination device, based on a mechanical battery for use with surgical robotic arms and/or carts. The mechanical battery is configured to perform a first function to mechanically stabilize the robotic arm and/or mobile cart to which the robotic arm is attached. In addition, the mechanical battery also performs a second function as an electrical battery to serve as a power supply (e.g., uninterrupted power supply (UPS)) providing electrical power to the robotic arm and/or mobile cart. This electrical battery function provides backup electrical power to the robotic arm and/or mobile cart in case power supply through cables is cut and/or eliminates the power cables altogether.

The position stability of a base of the robotic arm ensures the accurate movement of the robotic arm, including movement of a surgical instrument that is attached thereto. In particular, any change of the position and orientation (i.e., pose) of the base of the robotic arm transfers to the pose of an end-effector and position of the surgical instrument, and thus, impacts the accuracy of the procedure. Therefore, the supporting structures (e.g., the mobile cart) on which the robotic arm is mounted also should have high stability.

According to one embodiment of the present disclosure, a surgical robotic mobile cart is disclosed. The surgical robotic mobile cart includes a base having a plurality of supports (e.g., feet or wheels) and a robotic arm coupled to the base and configured to support at least one of a camera or an instrument. The mobile cart also includes a flywheel energy storage device coupled to the base. The flywheel energy storage device is configured to stabilize the base and to provide electrical energy.

Implementations of the above embodiment may include one or more of the following features. According to one aspect of the above embodiment, the flywheel energy storage device is operable in a first mode during which the flywheel energy storage device may be configured to convert electrical energy into kinetic energy and in a second mode during which the flywheel energy storage device may be configured to convert stored kinetic energy into electrical energy. According to other aspects of the above embodiment, the flywheel energy storage device may include: an axle defining a first rotation axis; a flywheel rotor coupled to the axle; and at least one induction device that may include a plurality of axial magnets and a plurality of radial magnets. The flywheel energy storage device may be configured to operate in a charging mode during which the radial magnets are energized to rotate the axle and the flywheel rotor. The flywheel energy storage device may be also configured to operate in a generating mode during which the plurality of the radial magnets output electrical energy in response to rotation of the plurality of axial magnets. The surgical robotic mobile cart may include a pair of diametrically opposing rotational joints coupled to the flywheel energy storage device. The pair of diametrically opposing rotational joints define a second rotation axis perpendicular to the first rotation axis. The pair of diametrically opposing rotational joints may be passive or active. Each of the diametrically opposing rotational joints may include an actuator. The surgical robotic mobile cart may include a tilt sensor configured to determine imbalance of the base or force sensors which measure the individual contact forces of the suspending elements (casters, wheel, or feet) against the floor. The pair of diametrically opposing rotational joints may be configured to rotate the flywheel energy storage device to adjust the imbalance of the base. The flywheel energy storage device may further include: a rotational sensor configured to measure a rotational velocity of the axle. The surgical robotic mobile cart may include a controller configured to calculate a charge level of the flywheel energy storage device based on the rotational velocity.

According to a further embodiment of the present disclosure, a surgical robotic mobile cart is disclosed. The surgical robotic mobile cart includes a base having a plurality of supports (e.g., feet or wheels) and a robotic arm coupled to the base and configured to support at least one of a camera or an instrument. The mobile cart also includes a flywheel energy storage device coupled to the base. The flywheel energy storage device is configured to stabilize the base and to provide electrical energy. The flywheel energy storage device includes: an axle defining a first rotation axis; a flywheel rotor coupled to the axle; and at least one induction device that may include a plurality of axial magnets and a plurality of radial magnets.

Implementations of the above embodiment may include one or more of the following features. According to one aspect of the above embodiment, the flywheel energy storage device may be configured to operate in a charging mode during which the radial magnets are energized to rotate the axle and the flywheel rotor, and in a generating mode during which the plurality of the radial magnets output to electrical energy in response to rotation of the plurality of axial magnets. The surgical robotic mobile cart may include a pair of diametrically opposing rotational joints coupled to the flywheel energy storage device. In one embodiment there could be a separate drive motor and a generator so that both modes run in parallel and without switching. The pair of diametrically opposing rotational joints define a second rotation axis perpendicular to the first rotation axis. The pair of diametrically opposing rotational joints may be passive or active. Each of the diametrically opposing rotational joints may include an actuator. The pair of diametrically opposing rotational joints may be configured to rotate the flywheel energy storage device to adjust the imbalance of the base. The flywheel energy storage device may further include a rotational sensor configured to measure a rotational velocity of the axle. The surgical robotic mobile cart may include a controller configured to calculate a charge level of the flywheel energy storage device based on the rotational velocity.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a mobile cart according to an embodiment of the present disclosure;

FIG. 2 is a perspective view of a surgical robotic arm of the surgical robotic system of FIG. 1 according to an embodiment of the present disclosure;

FIG. 3 is a perspective view of a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of mobile carts of FIG. 1 positioned about a surgical table according to an embodiment of the present disclosure;

FIG. 6 is a cross-sectional view of a flywheel energy storage device according to an embodiment of the present disclosure;

FIG. 7 is a perspective, schematic view of a flywheel rotor of the flywheel energy storage device according to an embodiment of the present disclosure; and

FIG. 8 is a bottom, schematic view of a base of the mobile cart with the flywheel energy storage device according to an embodiment of the present disclosure.

### DETAILED DESCRIPTION

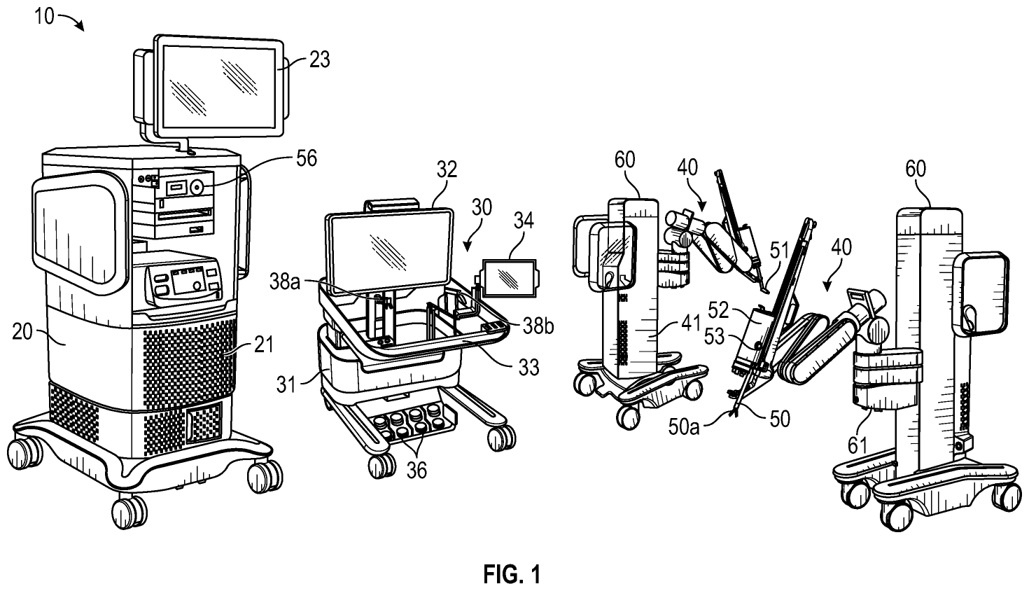

With reference to FIG. 1, a surgical robotic system 10 includes a control tower 20, which is connected to all of the components of the surgical robotic system 10 including a surgical console 30 and one or more movable carts 60. Each of the movable carts 60 includes a robotic arm 40 having a surgical instrument 50 removably coupled thereto. The robotic arms 40 is also coupled to the movable cart 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 perform the image processing based on the depth estimating algorithms of the present disclosure and output the processed video stream.

The surgical console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgical console 30 also includes a plurality of user interface devices, such as foot pedals 36 and a pair of handle controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

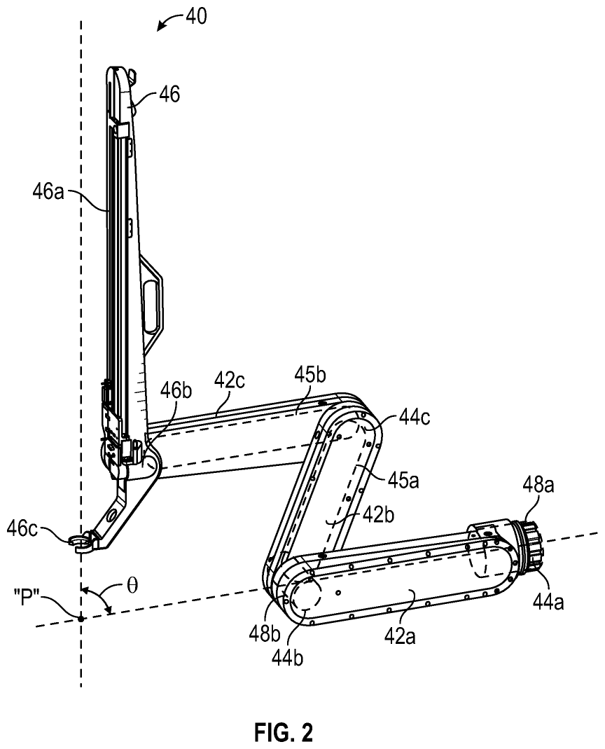

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the mobile cart 60 and defines a first longitudinal axis.

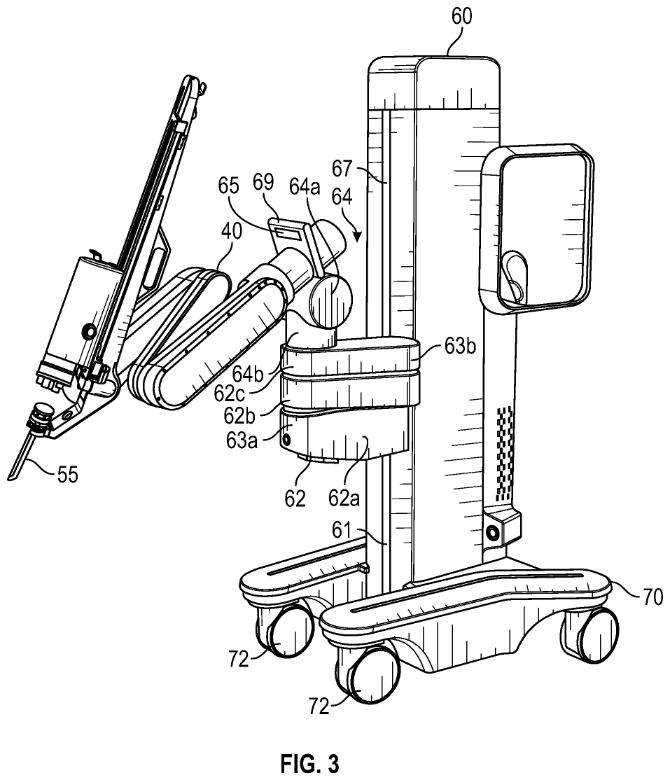

With reference to FIG. 3, the mobile cart 60 includes a lift 67 and a setup arm 61, which provides a base for mounting of the robotic arm 40. The lift 67 allows for vertical movement of the setup arm 61. The mobile cart 60 also includes a display 69 for displaying information pertaining to the robotic arm 40. In embodiments, the robotic arm 40 may include any type and/or number of joints. The mobile cart 60 includes a base 70 having a plurality of supports (e.g., feet or wheels) 72 allowing for movement of the mobile cart 60. The lift 67 extends vertically from the base 70.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic port 55 (FIG. 3) held by the holder 46. The holder 46 also includes a port latch 46c for securing the port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

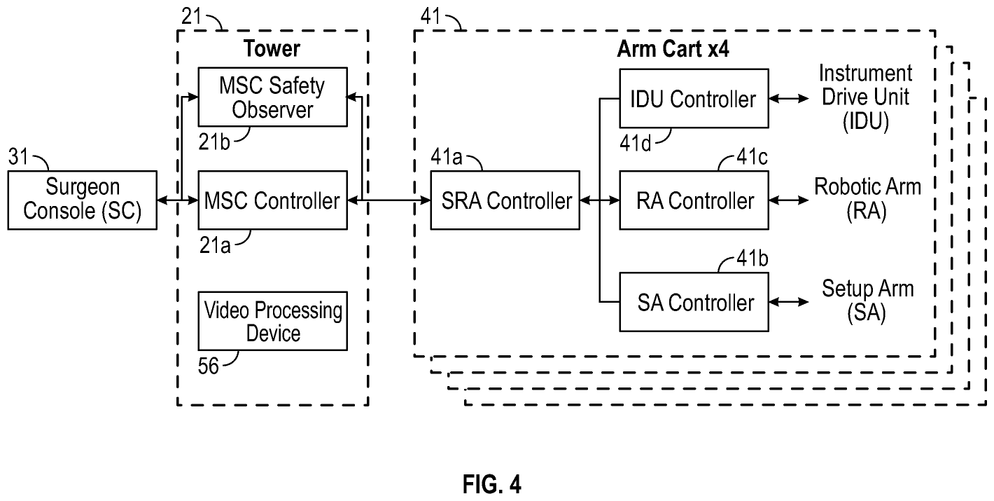

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgical console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the IDU 52. The main cart controller 41a also communicates actual joint angles back to the controller 21a.

The setup arm controller 41b controls each of joints 63a and 63b, and the rotatable base 64 of the setup arm 61 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controller 38a may be embodied as a coordinate position and roll-pitch-yaw (RPY) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

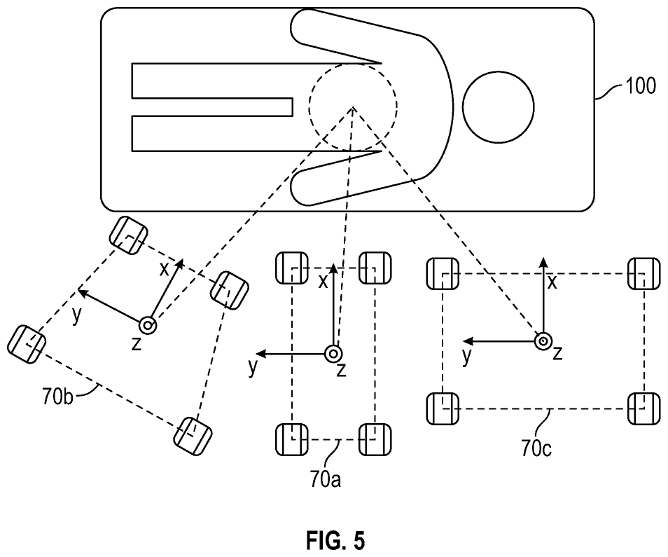

With reference to FIG. 5, the surgical robotic system 10 is setup around a surgical table 100. The system includes a plurality of mobile carts 60. The mobile cart 60 may include any suitably shaped and sized bases, e.g., one of the bases 70a-c, rectangular, trapezoidal, circular, oval, triangular, etc. The size and shape of the base 70 affects stability of the mobile cart 60, which also affects stability of the robotic arm 40. Thus, the larger the footprint of the base 70 or the distance between the supports 72, the more stable is the mobile cart 60. However, the footprint of the base 70 may also be based on the number of mobile carts 60 that need to be setup around the surgical table 100, i.e., within reach of the robotic arms 40 to the patient. The base 70a may have a narrower width than its length to allow for the mobile cart 60 to be oriented lengthwise and for positioning of multiple mobile carts 60 side-by-side along the surgical table 100. The base 70b has a trapezoidal shape, which also allows for fitting of multiple mobile carts 60 side-by-side, i.e., narrow (i.e., front) side being closer to the surgical table 100. The base 70c has a large footprint, which limits the number of mobile carts 60 being used with the surgical table 100. The base 70a, while being more desirable due to a smaller footprint than the bases 70b and 70c, may suffer from lack of stability, which limits the extensions of the robotic arm 40. Conversely, the bases 70b and 70c, while being more stable, are less desirable due to a larger footprint, which limits positioning of the mobile carts 60.

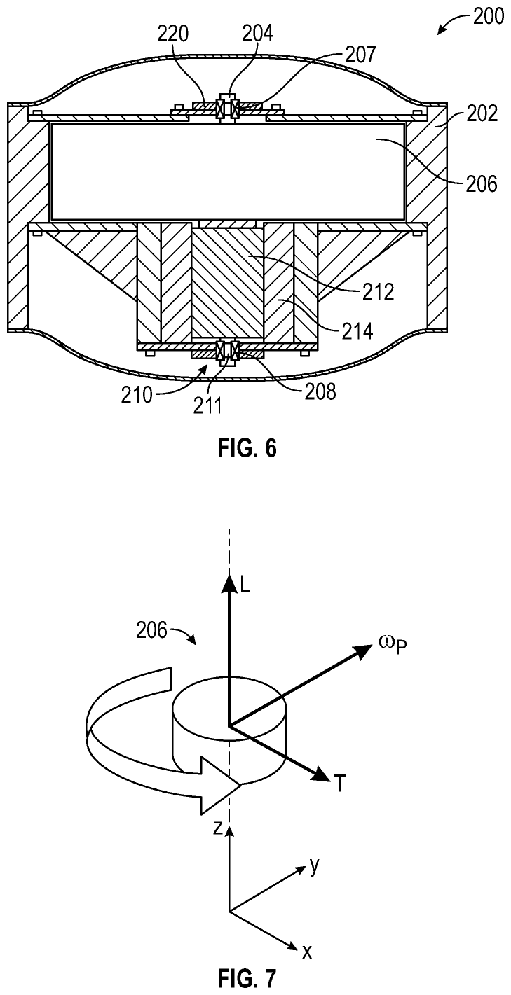

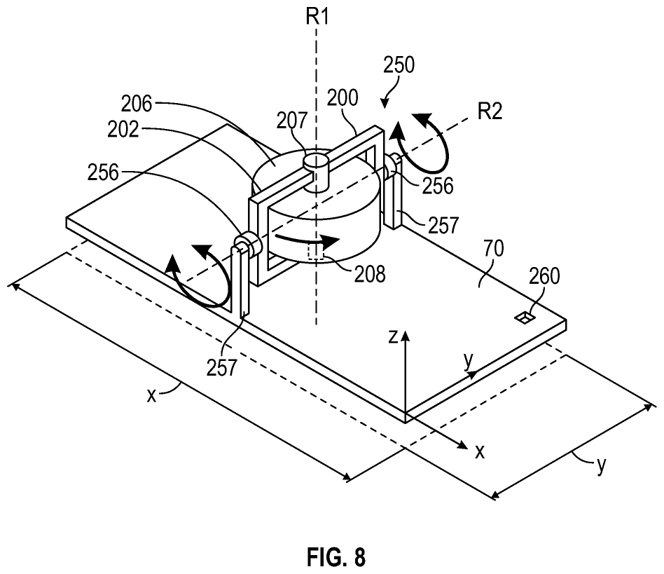

With reference to FIGS. 6 to 8, the present disclosure provides a flywheel energy storage (FES) device 200 which has two functions—stabilizing the base 70 while providing electrical power to the mobile cart 60 and the robotic arm 40. The FES device 200 is disposed in and/or below the base 70 (FIG. 8). The FES device 200 uses the gyroscopic effect to provide a stabilization torque to the mobile cart 60, thereby preventing tilting or tipping of the mobile cart 60 in any direction. The gyroscopic effect may be focused in any suitable direction allowing for modifying dimensions and shape of the footprint of the base 70, e.g., narrower side of the trapezoidal base 70b.

With reference to FIG. 6, the FES device 200 includes a housing 202 enclosing a central axle 204 with a flywheel rotor 206 attached to a first portion 204a of the axle 204. The flywheel rotor 206 has a substantially cylindrical shape and the axle 204 defines a first rotation axis R1 of the flywheel rotor 206. The flywheel rotor 206 may be formed from any suitable material, such as metal, carbon fiber, and combinations thereof. The axle 204 may be supported at each end within the housing 202 using bearings 207 and 208. The interior of the housing 202 may be under vacuum and the bearings 207 and 208 may be magnetic bearings to minimize air and rotational frictions, respectively.

The FES device 200 also includes an induction device 210, which acts as an electric motor configured to rotate the flywheel rotor 206 or as a generator rotated by the flywheel rotor 206 to generate electrical energy. The induction device 210 which includes an axial magnet 212 attached to a second portion 204b of the axle 204. The induction device 210 also includes a radial magnet 214. The axial and radial magnets 212 and 214 may be any suitable electromagnets used in electrical motor designs. During charging mode, the induction device 210 is operated as a motor and the radial magnet 214 is controlled (i.e., energized) to rotate axle 204. This allows the FES device 200 to convert inputted electrical energy into stored mechanical energy. During generating mode, the induction device 210 is operated as a generator and the radial magnets 214 generate electrical energy in response to rotation of the axial magnet 212. This allows the FES device 200 to convert stored mechanical energy into electrical energy. In an alternative embodiment a dedicated motor (to create kinetic energy) and a dedicated generator (to generate electric energy) are integrated to allow for parallel and seamless operation (no switching between modes).

With reference to FIG. 7, the flywheel rotor 206 is a rotating mass that is configured to store kinetic energy, which may be expressed using formula (I) below:

$$E {=}^{1} {/}_{2} \cdot / I / \cdot / ω {/}^{2} , \qquad ( I )$$

with I being the rotary inertia vector of the flywheel rotor 206 and @ the angular velocity vector. Assuming energy losses caused by friction or electrical inefficiencies (e.g., resistance), an electrical battery based on the FES device 200 does not degrade over life and monitoring its charge is more accurate than with electro-chemical batteries, which may be done by measuring the angular velocity of the flywheel rotor 206. The FES device 200 is configured to store kinetic energy for stabilizing the mobile cart 60 and preventing tipping, which allows for using narrower footprints increasing usability of multiple mobile carts.

Another aspect of the of the flywheel rotor 206 are gyroscopic effects (FIG. 7). A rotating flywheel rotor 206 with the inertia vector I rotating at an angular velocity vector op has an angular momentum, L, which may be expressed using formula (II) below:

$$L = I \times {ω}_{P} . \qquad ( II )$$

Whenever a torque T is applied to the flywheel (shown as a vector 7), the flywheel rotor 206 maintains its stability about this torque axis, while a pivot motion about the axis perpendicular to L and Tis induced (T=L×ωp). The angular momentum is sufficiently high so that when the required energy budget for powering the mobile cart 60 and/or the robotic arm 40 is fully drained, the flywheel rotor 206 retains sufficient speed to achieve stabilization. This may be achieved by monitoring rotational speed of the flywheel rotor 206 and preventing further reduction in rotational speed.

With reference to FIG. 8, a stabilization mechanism 250 is shown, which includes the FES device 200. While FIG. 8 shows that the stabilization mechanism 250 is attached to an underside of a rectangular base 70 (e.g., base 70a or 70c), the stabilization mechanism 250 may be used on any based having any suitable footprint, e.g., trapezoidal, rectangular, oval, circular, etc. In addition, the stabilization mechanism 250 may be disposed within the base 70, not just on its underside as shown.

The housing 202 is shown as a rectangular frame. The flywheel rotor 206 is rotationally coupled at the bearings 207 and 208, which are aligned with the first rotation axis R1 of the flywheel rotor 206. The orientation of the rotation axis R1 is perpendicular to the axis about which the mobile cart 60 is targeted to be stabilized (e.g., x-axis) and may be parallel with the z-axis

The FES device 200 may include multiple induction devices 210, one on top and one at the bottom, one of which may be used to provide kinetic energy to the flywheel rotor 206, while another harvests energy during the charging mode during (e.g., by speeding up the rotation of the flywheel rotor 206 as well as recuperating energy during the generating mode. The induction device 210 may also include one or more rotation sensors 211 (FIG. 6), which may be encoders, inertial measurement units (IMU), etc., to monitor the speed of rotation. The rotation sensor 211 is configured to provide rotational velocity measurement to the main cart controller 41a, which is configured to determine the charge of the FES device 200 using formula (I) as described above.

In further embodiments, one of the induction devices 210 (e.g., upper) may be used for acceleration (e.g., charging mode) of the flywheel rotor 206 whereas another induction device 210 (e.g., lower) may be used for recuperation (e.g., generating mode) of energy from the flywheel rotor 206, rather than operating a single induction device 210 in two modes (e.g., charging and generating). Dual induction device topology simplifies the electronics design. Furthermore, each of the induction devices 210 may different electrical characteristics (e.g., pole count) allowing for transforming voltage through the FES device 200.

The housing 202 is coupled to the base 70 using a pair of diametrically opposing rotational joints 256 defining a second rotation axis R2, which is perpendicular to the first rotation axis R1 and is parallel with a y-axis of the base 70. The joints 256 may attached to the base 70 using supports 257 and allow for rotation of the FES device 200 about the second rotation axis R2.

The joints 256 may be passive, e.g., bearings, or active, e.g., having actuators to actively control the angle of rotation about the second rotation axis R2. Active control of the joints 256 allows for targeting and countering tilting of the base 70. The base 70 may include one or more tilt sensors 260, which may be IMUs and the like, or force sensors (e.g., strain gauge sensors) disposed in one or more of the supports 72. The tilt sensors 260 are configured to measure imbalance in the base 70 and provide the signals to the main cart controller 41a, which then signals the joints 256 to rotate the FES device 200 to counteract the effects of the tilt. With the force sensors that measure the force distribution between the elements that contact the floor (wheels, casters, feet, etc.) a tipping force may be detected before tipping happens so that the actuators in the joint 256 generate a torque that counteracts the tipping force. This allows for adjustments before actual tipping occurs.

While the FES device 200 was described for use with the mobile cart 60, the FES device 200 may be also integrated into the robotic arm 40. In embodiments, the FES device 200 may be integrated the joint 40a of the robotic arm 40. This embodiment may be used where the robotic arm 40 is used without the mobile cart 60 and is attached to the surgical table 100, floor, ceiling, or any other surface or mount. This creates additional stability in any direction since the surgical table 100 or a ceiling/floor support systems may not have equal stiffness in all directions.

In certain embodiments, the speed of the flywheel rotor 206 may be lowered or fully stopped by short-circuiting the windings of the radial magnets 214. As shown in FIG. 6, the FES device 200 may also include a brake 220, which may be an electromagnetic friction brake configured to engage the axle 204 and/or the flywheel rotor 206. The brake 220 may be engaged to lower the speed of the flywheel rotor 206 more gradually.

Braking of the flywheel rotor 206 may be used in emergency situations where the stabilizing torque or remaining energy is no longer needed. Such braking may be automatically triggered by a certain software state of the system 10, loss of communication, or by user input. When braking is initiated, the joints 256 may rotate the FES device 200 to prevent orienting the FES device 200 that would destabilize the mobile cart 60 and/or the robotic arm 40. In addition, the torque needed to decelerate the flywheel rotor 206 may be monitored as well to prevent the torque being lower than a predetermined threshold, below which the mobile cart 60 may tip over. More specifically, the torque is controlled to avoid the first rotation axis R1 being parallel to the x-axis (FIG. 8). Thus, at the start of the emergency brake routine, the FES device 200 is initially rotated to align the first rotation axis R1 with the z-axis and then start to decelerate the flywheel rotor 206. At this point, suspending the brake torque would act about the z-axis which is safer as it would not lead to the tipping over of the mobile cart 60.

It will be understood that various modifications may be made to the embodiments disclosed herein. In embodiments, the sensors may be disposed on any suitable portion of the robotic arm. Therefore, the above description should not be construed as limiting, but merely as exemplifications of various embodiments. Those skilled in the art will envision other modifications within the scope and spirit of the claims appended thereto.

What is claimed is:

1. A surgical robotic mobile cart comprising:
a base having a plurality of supports;
a robotic arm coupled to the base and configured to support at least one of a camera or an instrument; and
a flywheel energy storage device coupled to the base, the flywheel energy storage device configured to stabilize the base and to provide electrical energy, the flywheel energy storage device operable in a first mode during which the flywheel energy storage device is configured to convert electrical energy into kinetic energy, and in a second mode during which the flywheel energy storage device is configured to convert stored kinetic energy into electrical energy.

2. A surgical robotic mobile cart comprising:
a base having a plurality of supports;
a robotic arm coupled to the base and configured to support at least one of a camera or an instrument; and
a flywheel energy storage device coupled to the base, the flywheel energy storage device configured to stabilize the base and to provide electrical energy.

3. The surgical robotic mobile cart according to claim 2, wherein the flywheel energy storage device includes:
an axle defining a first rotation axis;
a flywheel rotor coupled to the axle; and
at least one induction device including a plurality of axial magnets and a plurality of radial magnets.

4. The surgical robotic mobile cart according to claim 3, wherein the flywheel energy storage device is configured to operate in a charging mode during which the radial magnets are energized to rotate the axle and the flywheel rotor.

5. The surgical robotic mobile cart according to claim 3, wherein the flywheel energy storage device is configured to operate in a generating mode during which the plurality of the radial magnets output to electrical energy in response to rotation of the plurality of axial magnets.

6. The surgical robotic mobile cart according to claim 3, further comprising:
a pair of diametrically opposing rotational joints coupled to the flywheel energy storage device, the pair of diametrically opposing rotational joints defining a second rotation axis perpendicular to the first rotation axis.

7. The surgical robotic mobile cart according to claim 6, wherein the pair of diametrically opposing rotational joints are passive.

8. The surgical robotic mobile cart according to claim 6, wherein each of the diametrically opposing rotational joints includes an actuator.

9. The surgical robotic mobile cart according to claim 8, further comprising a tilt sensor configured to determine imbalance of the base.

10. The surgical robotic mobile cart according to claim 9, wherein the pair of diametrically opposing rotational joints are configured to rotate the flywheel energy storage device to adjust the imbalance of the base.

11. The surgical robotic mobile cart according to claim 3, wherein the flywheel energy storage device further includes:
a rotational sensor configured to measure a rotational velocity of the axle.

12. The surgical robotic mobile cart according to claim 11, further comprising a controller configured to calculate a charge level of the flywheel energy storage device based on the rotational velocity.

13. A surgical robotic mobile cart comprising:
a base having a plurality of supports;
a robotic arm coupled to the base and configured to support at least one of a camera or an instrument; and
a flywheel energy storage device coupled to the base, the flywheel energy storage device configured to stabilize the base and to provide electrical energy, the flywheel energy storage device includes:
an axle defining a first rotation axis;
a flywheel rotor coupled to the axle; and
at least one induction device including a plurality of axial magnets and a plurality of radial magnets.

14. The surgical robotic mobile cart according to claim 13, wherein the flywheel energy storage device is configured to operate in a charging mode during which the radial magnets are energized to rotate the axle and the flywheel rotor, and in a generating mode during which the plurality of the radial magnets output to electrical energy in response to rotation of the plurality of axial magnets.

15. The surgical robotic mobile cart according to claim 13, further comprising:
a pair of diametrically opposing rotational joints coupled to the flywheel energy storage device, the pair of diametrically opposing rotational joints defining a second rotation axis perpendicular to the first rotation axis.

16. The surgical robotic mobile cart according to claim 15, wherein the pair of diametrically opposing rotational joints are passive.

17. The surgical robotic mobile cart according to claim 15, wherein each of the diametrically opposing rotational joints includes an actuator.

18. The surgical robotic mobile cart according to claim 17, further comprising a tilt sensor configured to determine imbalance of the base, wherein the pair of diametrically opposing rotational joints are configured to rotate the flywheel energy storage device to adjust the imbalance of the base.

19. The surgical robotic mobile cart according to claim 13, further comprising a force sensor disposed in each support of the plurality of supports.

20. The surgical robotic mobile cart according to claim 13, wherein the flywheel energy storage device further includes:
a rotational sensor configured to measure a rotational velocity of the axle; and
a controller configured to calculate a charge level of the flywheel energy storage device based on the rotational velocity.
